(19) United States
(12) Patent Application Publication
(10) Pub. No.: US 2018/0244458 A1
(43) Pub. Date: Aug. 30, 2018

(54) SYSTEM AND METHOD FOR DISPENSING A NITRIC OXIDE SOLUTION FROM A VESSEL

(71) Applicants: Kori Dunn, Buffalo, NY (US); Lloyd A. Brown, Amherst, NY (US); Paul K. Oetinger, Gasport, NY (US)

(72) Inventors: Kori Dunn, Buffalo, NY (US); Lloyd A. Brown, Amherst, NY (US); Paul K. Oetinger, Gasport, NY (US)

(21) Appl. No.: 15/891,640

(22) Filed: Feb. 8, 2018

Related U.S. Application Data
(60) Provisional application No. 62/464,456, filed on Feb. 28, 2017.

(51) Int. Cl. B65D 83/14; B65D 83/00; A61L 2/00; A61L 2/18; A61L 2/26

(52) U.S. Cl. CPC B65D 83/14; B65D 83/0022; A61L 2/26; A61L 2/18; A61L 2/0088

(57) ABSTRACT

A system and method for dispensing a nitric oxide solution, and more preferably a gas/liquid mixture of nitric oxide in a solvent, from a cylinder or other containment vessel are provided. The dispensing system and method are configured so as to maintain the nitric oxide based solution within the cylinder or containment vessel at a constant pressure above ambient pressure as the gas/liquid mixture is stored in and depleted from the containment vessel thus preventing the nitric oxide from coming out of the solution. In addition, a method of producing the gas/liquid mixture of nitric oxide in a solvent, such as deoxygenated and deionized water, as well as a cylinder filling method and apparatus are also provided.

### CROSS REFERENCE TO RELATED APPLICATIONS

The present application claims priority from U.S. Provisional Patent Application Ser. No. 62/464,456 filed on Feb. 28, 2017; the disclosure of which is incorporated by reference herein.

### TECHNICAL FIELD

The present invention relates to a system and method for dispensing a nitric oxide solution from a vessel, and more particularly, to a system and method for dispensing a gas/liquid mixture of nitric oxide in a liquid solvent from a multi-chamber containment vessel. Still more particularly, the present invention relates to a system and method for dispensing a gas/liquid mixture of nitric oxide in a liquid solvent from a containment vessel that maintains the gas/liquid mixture within one of the chambers within the containment vessel at a prescribed pressure range above ambient pressure as the volume of the gas/liquid mixture in the vessel changes and/or prevents the nitric oxide from coming out of the liquid solvent within the vessel.

### BACKGROUND

There have recently been developed various systems and methods for sanitizing or disinfecting the surface of a body part or item using a nitric oxide solution. One example of such system and method is described in U.S. Patent Application Publication No. 2014/0186211. A preferred nitric oxide solution that is useful with such system and method is a gas/liquid mixture of nitric oxide in deionized and deoxygenated water. The challenge in using nitric oxide solutions in such sanitation and disinfection systems is the degradation of the nitric oxide solution over time as contaminants may infiltrate the nitric oxide solution prior to dispensing. Another problem associated with such nitric oxide solutions, and in particular the gas/liquid mixture of nitric oxide in a liquid solvent, is that the nitric oxide gas may come out of the liquid solvent during storage, transport and dispensing of the gas/liquid mixture resulting inconsistent nitric oxide concentration in the dispensed mixture.

What is needed, therefore, is a containment vessel for a gas/liquid mixture comprising nitric oxide in a liquid solvent configured to be operably integrated into such nitric oxide based sanitation and disinfection systems and associated methods. The containment vessel must prevent infiltration of contaminants into the gas/liquid mixture and must be capable of maintaining a substantially constant nitric oxide concentration in the liquid solvent as the solution is stored within the vessel and, more importantly, as the solution is dispensed from the vessel and thus depleted over time.

### SUMMARY OF THE INVENTION

The present invention may be characterized as a vessel for a gas/liquid mixture comprising nitric oxide in a liquid solvent, the vessel comprising: (i) a housing having a body section, a dispensing end, and an opposite end, the body section defining an interior chamber having one or more interior surfaces; and (ii) a moveable or expandable structure having a distal end, a proximal end, and one or more surfaces, the moveable or expandable structure disposed within the interior chamber to define a first section of the interior chamber configured to hold the gas/liquid mixture of nitric oxide in liquid solvent and a second section of the interior chamber configured not to hold any portion of the gas/liquid mixture of nitric oxide in liquid solvent. The dispensing end of the housing includes at least one dispensing port through which the gas/liquid mixture of nitric oxide in liquid solvent exits the first section of the interior chamber as the moveable or expandable structure moves or expands and at least one of the one or more surfaces of the moveable or expandable structure remains in contact with a liquid surface of the gas/liquid mixture of nitric oxide in liquid solvent in the first section of the interior chamber to prevent nitric oxide from coming out of the liquid solvent.

Alternatively, the present invention may be characterized as a method of producing a gas/liquid mixture of nitric oxide in deionized and deoxygenated water comprising the steps of: (a) purifying a source of water; (b) deionizing the water; (c) deoxygenating the deionized water; (d) dissolving nitric oxide into the deionized and deoxygenated water; (e) rinsing/purging a plurality of cylinders, cartridges or other containment vessels; and (f) filling a plurality of cylinders, cartridges or other containment vessels with the gas/liquid mixture of nitric oxide in deionized and deoxygenated water.

Still further, the present invention may be characterized as a dispensing system for a gas/liquid mixture comprising: (i) a container comprising a body section defining an interior chamber having one or more interior surfaces; a dispensing end; and an opposite end; (ii) a moveable or expandable structure disposed within the interior chamber of the container, the moveable or expandable structure having a distal end, a proximal end, and one or more side surfaces, the moveable or expandable structure configured such that the one or more side surfaces are sealingly engaged with the one or more interior surfaces of the interior chamber of the container to define a first section of the interior chamber between to the distal end of the moveable or expandable structure and dispensing end of the container and a second section of the interior chamber between to the proximal end of the moveable or expandable structure and the opposite end of the container; (iii) the gas/liquid mixture comprising nitric oxide gas in liquid solvent disposed in the first section of the interior chamber of the container; and (iv) a motive source in operative association with the container and the moveable or expandable structure and configured to cause the moveable or expandable structure to traverse the interior chamber of the container. The dispensing end of the container includes at least one port through which the gas/liquid mixture of nitric oxide in liquid solvent is dispensed as the moveable or expandable structure traverses or expands within the interior chamber of the container and the distal end of the moveable or expandable structure remains in contact with a liquid surface of the gas/liquid mixture of nitric oxide in liquid solvent in the first section of the interior chamber so as to prevent nitric oxide from coming out of the liquid solvent.

### BRIEF DESCRIPTION OF THE DRAWINGS

While the present invention concludes with claims distinctly pointing out the subject matter that Applicants regard as the invention, it is believed that the invention will be better understood when taken in connection with the accompanying drawings in which:

FIG. 1 is a cross-section view of a containment vessel for a liquid nitric oxide solution such as a gas/liquid mixture comprising nitric oxide in a liquid solvent in accordance with an embodiment of the present invention incorporating a moveable element disposed within the containment vessel;

FIG. 2 is a cross-section view of a containment vessel for a liquid nitric oxide solution such as a gas/liquid mixture comprising nitric oxide in liquid solvent in accordance with another embodiment of the present invention incorporating a moveable element disposed within the containment vessel;

FIG. 3 is a cross-section view of a containment vessel for a liquid nitric oxide solution such as a gas/liquid mixture comprising nitric oxide in liquid solvent in accordance with still another embodiment of the present invention incorporating a moveable element disposed within the containment vessel;

FIG. 4 is a cross-section view of a containment vessel for a liquid nitric oxide solution such as a gas/liquid mixture comprising nitric oxide in liquid solvent in accordance with a further embodiment of the present invention incorporating an expandable element disposed within the containment vessel;

FIG. 5 is a perspective view of a cylinder or vessel filling station suitable for use in the cleaning and filling of cylinders or other vessels with nitric oxide containing solution;

FIG. 6 is a top plan view of the cylinder or vessel filling station of FIG. 5; and

FIG. 7 is a schematic representation of a system for producing nitric oxide in deionized and deoxygenated water.

### DETAILED DESCRIPTION

### Containment Vessel for Nitric Oxide Solutions

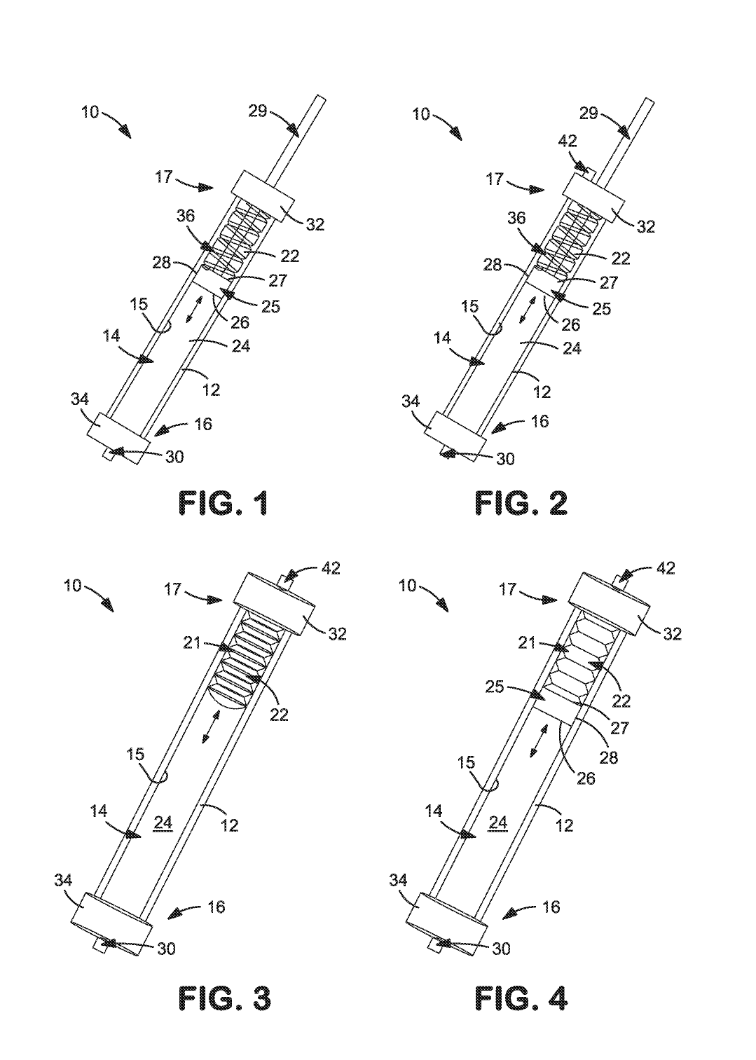

Turning now to FIGS. 1, 2 and 3, there is shown a containment vessel 10 configured to hold a liquid nitric oxide solution such as a gas/liquid mixture comprising nitric oxide in liquid solvent. The containment vessel 10 comprises a generally cylindrical housing 12, a moveable structure 20 disposed within the housing 12; a dispensing port 30, and one or more filling ports 40, 42. In the illustrated embodiment, the housing 12 includes a body section 13 defining an interior chamber 14 with one or more interior surfaces 15, a dispensing end 16, an opposite end 17. Interior chamber 14 is partitioned by the moveable structure 20 into a first section 22 configured to hold the gas/liquid mixture comprising nitric oxide in liquid solvent and a second section 24 configured that is sealably segregated from the first section 22 and does not contain or hold any portion of the gas/liquid mixture comprising nitric oxide in liquid solvent.

In the illustrated embodiments of FIGS. 1 through 3, the moveable structure is a piston 25 that traverses the interior chamber 14 of the housing 12. The piston includes a distal end 26, a proximal end 27, and one or more side surfaces 28 disposed within the interior chamber such that the one or more side surfaces 28 are sealingly engaged with the one or more interior surfaces 15 of the interior chamber 14. The location and positioning of the piston 25 defines the first section 22 of the interior chamber 14 between the distal end 26 of the piston 25 and the dispensing end 16 of the housing 12 and the second section 24 of the interior chamber 14 between to the proximal end 27 of the piston 25 and the opposite end 17 of the housing 12. The actuating force to move to piston 25 in a downward direction toward the dispensing end 16 may be a spring 36 (e.g. a constant force spring), a pneumatic force or hydraulic force applied to the proximal end of the piston 25, or to an expandable bellows that moves piston 25, or other electrical or mechanical force such as a motor (not shown) connected to the piston 25 via piston rod 29. Where a pneumatic or hydraulic force is used to move the piston 25 or piston/bellows arrangement within the interior chamber as depicted in FIG. 2 and FIG. 3, a pneumatic or hydraulic filling port is located proximate opposite end 17 of the housing 12 in fluid communication with the second section 24 of interior chamber 14.

The dispensing end 16 of the housing 12 includes at least one dispensing port 30 through which the gas/liquid mixture of nitric oxide in liquid solvent exits the first section 22 of the interior chamber 14 and is dispensed as the piston 25 moves within the interior chamber 14 toward the dispensing end 16 of the housing 12. In addition, a filling port configured to add the gas/liquid mixture of nitric oxide in deionized and/or deoxygenated water to the first section 22 of the interior chamber is disposed on the body section 13 of the housing 12 and preferably located proximate the dispensing end 16. In some embodiments, the filling port and dispensing port may be the same.

The preferred embodiment of the containment vessel 10 is constructed from a high quality 316 stainless steel (316SS). This common grade of stainless steel is preferred for applications of nitric oxide in in liquid solvent because of its corrosion resistance properties. At the bottom of the housing 12 proximate dispensing end 16 a 316SS bottom cap 34 is disposed. This bottom cap 34 contains a connection for a standard 316SS ball valve (not shown). At the top of the housing 12 proximate the opposite end 17, a 316SS top cap 32 is disposed. The top cap 32 preferably contains an opening 33 for the piston rod 29 and connections for a pressure gauge (not shown) and an outlet valve (not shown), such as a standard 316SS ball valve. The ball valves may be attached to 316SS quick connects for ease of use. The piston 25 is also preferably a 316SS piston assembly with a connection for the piston rod 29, also made of 316SS, and a groove (not shown) for a gasket or O-ring (not shown). The gaskets and O-rings are used to ensure that the piston 25 maintains pressure-tight contact with the interior surface 15 of the housing 12 and to prevent contaminants from entering from the second section 24 of the interior chamber 14 to the liquid/gas mixture within the first section 22 of the interior chamber 14. Additional gaskets (not shown) may be placed between the housing 12 and top cap 32; between the housing 12 and bottom cap 34; and between the piston rod 29 and top cap 32. A 316SS retainer may also be used to hold the O-ring in place between the piston rod 29 and the top cap 32 to maintain pressure-tight seals.

Alternative materials suitable for use in the construction of the containment vessel and components shown in the Figs. include any materials generally compatible with nitric oxide. Examples of such materials: include carbon steel, stainless steel, copper, aluminum, monel alloys, fluorocarbon based polymers (e.g. Kel-F, Teflon, Tefzel, Kynar) polyvinylchloride, polycarbonates, and various fluoropolymer elastomers including those sold under the trade names Kalrez and Viton. However, it should be understood that nitric oxide can be corrosive to certain metals such as aluminum, carbon steel, and low quality stainless steels when in the presence of water.

The arrangements depicted in FIGS. 1 through 5 are configured such that at least one of the one or more surfaces of the moveable or expandable structure remains in contact with the liquid surface of the gas/liquid mixture of nitric oxide in liquid solvent in the first section of the interior chamber so as to prevent nitric oxide from coming out of the liquid solvent. Such arrangements also maintain the gas/liquid mixture of nitric oxide in liquid solvent in the first section at a prescribed pressure range above ambient pressure as the volume of the gas/liquid mixture within the first section of the interior chamber changes by virtue of the gas/liquid mixture of nitric oxide in liquid solvent being dispensed from the vessel. Finally, such arrangements also sealably isolate the gas/liquid mixture of nitric oxide in liquid solvent in the first section of the interior chamber from any contaminants present in the second section of the interior chamber or from outside the container

In the embodiment of FIG. 1, a spring is used to ensure that the piston maintains contact with the surface of the gas/liquid mixture during both storage and use. In this embodiment, the length of the spring when it is fully decompressed must be greater than or equal to the length of the housing, such that the spring is capable of driving the piston all the way to the dispensing end of the housing while keeping the distal end of the piston in contact with the surface of the gas/liquid mixture and keeping the gas/liquid mixture within a prescribed pressure range above ambient pressure so as to prevent nitric oxide from coming out of the liquid solvent.

In the embodiment of FIG. 2, the force on the piston required to maintain the gas/liquid mixture in the first section at a prescribed pressure range above ambient pressure and to keep the distal end of the piston in contact with the surface of the gas/liquid mixture is applied by both a spring and a pneumatic/hydraulic force. When the first section of the interior chamber vessel is full of the gas/liquid mixture, i.e. during storage and transport, the spring acts to provide the requisite force on the proximal end of the piston. Then, during use, the containment vessel is connected to either a compressed gas source or hydraulic fluid source via the filling port that is in fluid communication with the second section of the interior chamber. As the gas/liquid mixture of nitric oxide in liquid solvent is dispensed, the pneumatic force from the compressed gas or the hydraulic force from the fluid force the piston in a downward direction to keep the distal end of the piston in contact with the surface of the gas/liquid mixture and keeps the gas/liquid mixture within a prescribed pressure range above ambient pressure so as to prevent nitric oxide from coming out of the liquid solvent.

The use of a spring together with a compressed gas or liquid source makes sense because the energy stored in the spring can be used to maintain the gas/liquid mixture within the first section of the interior chamber within a prescribed pressure range above ambient pressure during storage and transport when no energy is available in the form of a compressed gas or liquid. Then, when the vessel is connected or inserted into a delivery apparatus, a compressed gas or liquid source is available to maintain the force on the piston constant during use.

In the embodiment of FIG. 3, the force on the piston required to maintain the gas/liquid mixture in the first section at a prescribed pressure range above ambient pressure and to keep the distal end of the piston in contact with the surface of the gas/liquid mixture is applied by a moveable bellows connected to the proximal end of the piston and fluidically coupled to either a compressed gas source or hydraulic fluid source via the filling port. As the gas/liquid mixture of nitric oxide in liquid solvent is dispensed, the pneumatic force from the compressed gas or the hydraulic force from the fluid expand the bellows causing the piston structure to move in a downward direction keeping in contact with the surface of the gas/liquid mixture and keeping the gas/liquid mixture within a prescribed pressure range above ambient pressure so as to prevent nitric oxide from coming out of the liquid solvent.

The piston assembly proposed in the embodiments of FIGS. 1 through 3 also simplifies filling of the vessels with the gas/liquid mixture of nitric oxide in liquid solvent. Since, it is important that the interior chamber of the vessel be deoxygenated before the gas/liquid mixture of nitric oxide in liquid solvent is added to the interior chamber to avoid the nitric oxide reacting with oxygen to form acidic byproducts, the movement of the piston to the dispensing end of the housing aids keeping the interior chamber of the vessel generally oxygen-free.

For example, when the piston is lowered all the way to the dispensing end of the housing, the first section of the interior chamber will be completely evacuated and generally oxygen-free. The vessel can then be filled with the gas/liquid mixture of nitric oxide in liquid solvent from the dispensing port or alternatively from the filling port located proximate the dispensing end of the housing. Prior to filling the first section of the vessel with the gas/liquid mixture of nitric oxide in liquid solvent, it may be beneficial to pull a vacuum or partial vacuum in the first section so as to ensure there is little or no oxygen present in the first section of the interior chamber before filling.

In the embodiments of FIG. 4 a bellows or a bladder can be used to maintain the first section of the interior chamber head space free during storage and/or use. In these arrangements, a bellows or bladder may be in direct fluid contact with the gas/liquid mixture of nitric oxide in liquid solvent in the interior chamber of the vessel housing. The second section of the interior chamber corresponds to the interior volume of the bellows or bladder. As the gas/liquid mixture of nitric oxide in liquid solvent is withdrawn from the vessel via the dispensing port, a compressed gas or another fluid enters the interior volume of the bellows or bladder via the port at the opposite end of the housing causing it to expand and thus increasing the volume of the second section (i.e., interior volume of the bellows or bladder) and correspondingly decreasing the volume of the first section. As the bellows or bladder expands, the outer surface maintains contact with the surface of the gas/liquid mixture and keeps the pressure of the gas/liquid mixture of nitric oxide in liquid solvent in the first section of the interior chamber within the prescribed pressure range above ambient pressure.

Knowing the location or position of the piston, piston rod, or bellows/bladder allows one to ascertain or calculate the volume of gas/liquid mixture remaining in the containment vessel and therefore to determine when the gas/liquid mixture is depleted or how many further dispenses or doses remain in the vessel prior to replacing, refilling or changing the containment vessel. For example, the location or position of the piston within the interior chamber with respect to a reference point on the interior surface of the interior chamber can be used to determine the volume of gas/liquid mixture remaining in the containment vessel. Alternatively, the position or location of the piston rod or the bellows/bladder with respect to a reference point can also be used to determine the volume of solution remaining in the containment vessel. In one embodiment, the movement and location of the piston rod relative to the reference point can be discerned using a shaft roller and a potentiometer attached to the top cap using a bracket.

### Nitric Oxide Dispensing System, Electronic Controls, Display and Alarms

One embodiment of a dispensing system for sanitizing or disinfecting the surface of a body part or item using a liquid nitric oxide solution is shown and described in U.S. Patent Application Publication No. 2014/0186211, the disclosure of which is incorporated by reference herein. Other embodiments of the liquid nitric oxide solution dispensing systems contemplated for use with the presently disclosed containment vessel include stationary liquid nitric oxide solution dispensing systems that are affixed to a wall, a table or free-standing units. Alternatively, the liquid nitric oxide solution dispensing systems may be configured as portable dispensing systems disposed on a cart and fluidically coupled to one or more containment vessels also disposed on the cart. Such portable dispensing systems might typically include a wheeled cart that can accommodate one or more containment vessels of various sizes and types the liquid nitric oxide solution (of various sizes and types) and a surface that allows for mounting of the dispensing system and accessories. The wheeled cart could readily be moved to multiple locations within a facility where such sanitation and disinfection is required.

The typical liquid nitric oxide solution dispensing system would be configured to operate with pressurized containment vessels at pressures between about 0 psig and 1500 psig, and more preferably at pressures between about 5 psig and 100 psig. The size of the containment vessel is preferably between about 0.1 liters and 200 liters, and more preferably between about 0.5 liters and 4.0 liters. Containment vessels with internal volumes greater than about 20.0 liters would likely be useful for storage and transport of the liquid nitric oxide solutions and not for integration with a dispensing system. The dispensing volume or dosing volume of the liquid nitric oxide solution, such as the gas/liquid mixture of nitric oxide in the liquid solvent, is preferably between about 3 ml to 100 ml, and more preferably about 10 ml. Lastly, the temperature range for the storage and transport of containment vessels of nitric oxide in liquid solvent is between about −40° C. to 65° C., and more preferably below about 25° C. To increase the stability of nitric oxide in the liquid solvent, the containment vessels may be refrigerated to temperatures below about 20° C.

Various embodiments of the liquid nitric oxide solution dispensing system or the present containment vessel preferably include an electronic display capable of displaying information in digital and/or analog form and an electronic alarm capable of being actuated to signify the presence of an alarm state audibly, visibly, or both audibly and visibly during the use of the vessel and associated dispensing system. In such embodiments the system may also include an electronic control processor connected to the various sensors, the electronic display, and the electronic alarm.

The electronic control is preferably a microprocessor based controller configured to receive one or more signals from the plurality of sensors as well as user inputted data or transmitted data signifying vessel contents, location, user information, and/or other conditions for use of the containment vessel or liquid nitric oxide dispensing system. The data and information are processed by the electronic control with selected parameters and information being concurrently displayed on the electronic display, including for example, the pressure within the vessel and the number of dispensing doses remaining in the vessel.

The electronic control is preferably configured to generate a signal actuating the electronic alarm when the volume of the nitric oxide liquid solution in the vessel reaches a preset or predetermined value suggesting the vessel is in an empty or almost empty condition and the vessel needs to be replaced. Use of the dispensing device when the vessel is empty or near empty (e.g. fewer than a prescribed number of dispensing dosages remaining) may lead to inadequate disinfection of the surfaces to be treated. In applications using a gas/liquid mixture of nitric oxide in a liquid solvent, an audible or visual alarm or indicator may also be activated when a dose is dispensed. Such dispensing alarm or indicator is useful because a user may not realize whether or not any solution was actually dispensed, as the dispensing system often aerosolizes the gas/liquid mixture prior to contact with the surfaces to be sanitized or disinfected. Thus, the dispensing alarm or indicator confirms to the user that the solution was dispensed.

The electronic control is further configured to generate a signal actuating the electronic alarm when the pressure of the liquid nitric oxide solution in the vessel reaches a preset or predetermined low threshold. In order to ensure the dispensed solution likely has sufficient nitric oxide necessary to achieve the desired microbial kill, the pressure of the nitric oxide liquid solution in the vessel needs to above a prescribed threshold pressure. If the pressure in the vessel is somehow reduced to a value at or below the low pressure threshold, there may be a leak in the system or the nitric oxide may come out of solution thus rendering the dispensed solution ineffective for sanitation and disinfection purposes. Use of a low pressure alarm identifies conditions when the dispensing device should not be used and the vessel or dispensing system should be replaced.

The electronic display can be configured to display either graphical information or numerical information, or both. Any useful information can be displayed, such as: the pressure of gas/liquid mixture in the containment vessel; the amount of gas/liquid mixture remaining in the cylinder or containment vessel; the number of dispensing doses remaining until the amount of solution remaining in the cylinder or containment vessel is low enough to reach a predetermined threshold value or to be completely exhausted from the cylinder or containment vessel; the status (including alarm status) of the cylinder or containment vessel, or other desired information. The format of the display can take the form of a symbol that comes on or flashes, an analog scale and/or a digital display, or other formats. The display can be activated so that different items of information appear together, or alternatingly (i.e. with one item appearing, then a second item, then the first again, and so on).

The disclosed containment vessel and liquid nitric oxide solution dispensing system may also be fitted with additional sensors such as a chemical sensor, a temperature sensor, light sensor, accelerometer, magnetic field sensing, etc. to provide additional functionality for the device. A device incorporating a chemical sensor would be useful to detect impurities or presence of toxic gases and/or to assure the proper gas composition is delivered from the cylinder during use, particularly medical gas uses. For example, a built-in chemical sensor may be used to determine if effluents from the containment vessel or the dispensing device are safe and/or meet the required quality targets prior to any further use/dispensing. This is of critical importance in situations where toxic gases such as nitrogen dioxide may be present.

Other sensors that could be incorporated within the disclosed liquid nitric oxide solution dispensing system and/or containment vessel may include the capability to sense proximity to a magnetic field of a given strength or the ability to detect temperature excursions outside of specific window and disable the device or emit an alarm if such temperature excursions or presence of magnetic fields pose a safety hazard. Another type of sensor suitable for integrating into the containment vessel might include an accelerometer to monitor/assure proper handling of the vessel or emit an alarm if the cylinder had been dropped or excessively mishandled during transport. Yet another sensor or detecting means that monitors the leak integrity of the containment vessel and/or dispensing system during an off state which and alerts the user that a leak may be present within the system. Such leak integrity sensors may be atmosphere monitoring sensors, chemical sensors, and/or pressure sensing techniques.

A particularly advantageous feature of the present dispensing system and/or containment vessel would be the ability to transmit and/or receive data to and/or from different external sources, such as a smartphone. For example, a global positioning system or GPS type chip may be integrated within the containment vessel and/or dispensing system to provide functionality regarding location or inventory tracking of the vessels. In addition, electronic transmission of the vessel contents to a data storage or central processing unit is contemplated for purposes of usage tracking, cylinder replacement planning, and other administrative or logistical functions. In medical applications, such communications would preferably be compliant or example with ISO/IEEE 11073-30300, “Health informatics—Point-of-care medical device communication—Part 30300, et seq. Such wireless communications incorporated within the device would preferably be configured with power saving features, so as to minimize power usage and preserve the battery life.

Certain embodiments of the present containment vessel and/or dispensing system preferable incorporate wireless or hard-wired communication features and, in particular, a data receiving capability which would allow use of externally supplied data to assist in the dispensing system. Wireless or hard wired communications can also be part of the security features of the present device. For example, operation of the device may be authorized through the use of RFID fobs, barcode scanning, or chip technology to identify an authorized user of the gas cylinder and device. The device may also incorporate anti-tampering features to identify situations where an unauthorized use of the device and release of the cylinder contents is attempted. An alternative arrangement would couple the present containment vessel and/or dispensing system to an external device which contains the communication and/or sensing functions and features described above.

### Liquid Based Nitric Oxide Solutions

The present invention relates to applications involving the use of a liquid based nitric oxide solution for sanitation and disinfection purposes. The liquid based nitric oxide solution is preferably a gas/liquid mixture comprising a liquid solvent containing gaseous nitric oxide dispersed therein. The liquid solvent is preferably water, alcohols, or mixtures thereof, and most preferably comprises deionized and deoxygenated water.

The nitric oxide solution provided in embodiments disclosed herein may contain one or more type of additives such as preservatives, surfactants, solution stabilizers, anti-microbial agents, pH adjusters, or an agent that is capable of accelerating or inhibiting evaporation. As disclosed in U.S. Patent Application Publication No. 2014/0186211, certain additives that are preservatives may include butylated hydroyanisole (BHA), butylated hydroxytoluene (BHT), benzoic acid, ascorbic acid, methyl paraben, propyl paraben, tocopherols and mixtures thereof. Additives such as ammonia may be used to alkalinize the solution, or hydrochloric acid to acidify the solution.

As further disclosed in U.S. Patent Application Publication No. 2014/0186211, certain additives may include ethylene glycol or polyethylene glycol or other anti-microbial agents other than nitric oxide. Some of the other anti-microbial additives that may be used include but are not limited to halogenated aromatics, chlorinated hydrocarbons, organometallics, metallic salts, organic sulfur compounds, quaternary ammonium compounds, phenolics, triclosan, 3,4,4′-trichlorocarbanilide (triclocarban), 3,4,4′-trifluoromethyl-4,4′-dichlorocarbanilide (cloflucarban), 5-chloro-2-methyl-4-isothiazolin-3-one, iodopropynlbutylcarbamate, 8-hydroxyquinoline, 8-hydroxyquinoline citrate, 8-hydroxyquinoline sulfate, 4-chloro-3,5-xylenol(chloroxylenol), 2-bromo-2-nitropropane-1,3-diol, diazolidinyl urea, butylparaben, ethylparaben, methylparaben, methylchloroisothiazoline, methylisothiazoline, a mixture of 1,3-bis(hydroxymethyl)-5,5-dimethylhydantoin and 3-iodo-2-propynyl butyl carbamate, oxyquinoline, EDTA, tetrasodium EDTA, p-hydroxyl benzoic acid ester, alkyl pyridinum compounds, coco phosphatidyl PG-dimonium chloride, chlorhexidine gluconate, chlorhexidine digluconate, chlorhexidine acetate, chlorhexidine isethionate, chlorhexidine hydrochloride, benzalkonium chloride, benzethonium chloride, polyhexamethylene biguanide, and zinc salt, or mixtures thereof.

### Production of Nitric Oxide Solution and Cylinder Filling

Production and manufacture of liquid based nitric oxide solutions, and more particularly a gas/liquid mixture of nitric oxide in deionized and deoxygenated water is preferably done in a manner that avoids or minimizes formation of acidic byproducts. Broadly speaking, the preferred method of producing a gas/liquid mixture of nitric oxide in deionized and deoxygenated water involves the following steps: (1) purifying a source of water; (2) deionizing the water; (3) deoxygenating the water; (4) dissolving nitric oxide into the deionized and deoxygenated water; (5) rinsing/purging a plurality of cylinders, cartridges or other containment vessels; and (6) filling a plurality of cylinders, cartridges or other containment vessels with the gas/liquid mixture of nitric oxide in deionized and deoxygenated water. Appropriate rinsing and purging of the associated tanks, piping, valves, and cylinders or containment vessels are encouraged to avoid the formation of acidic byproducts resulting from reaction of nitric oxide and/or nitrogen dioxide. The entire system and process should be configured to ensure that the gas/liquid mixture of nitric oxide in deionized and deoxygenated water remains pressurized throughout the process, as the nitric oxide will come out of solution almost immediately upon depressurization.

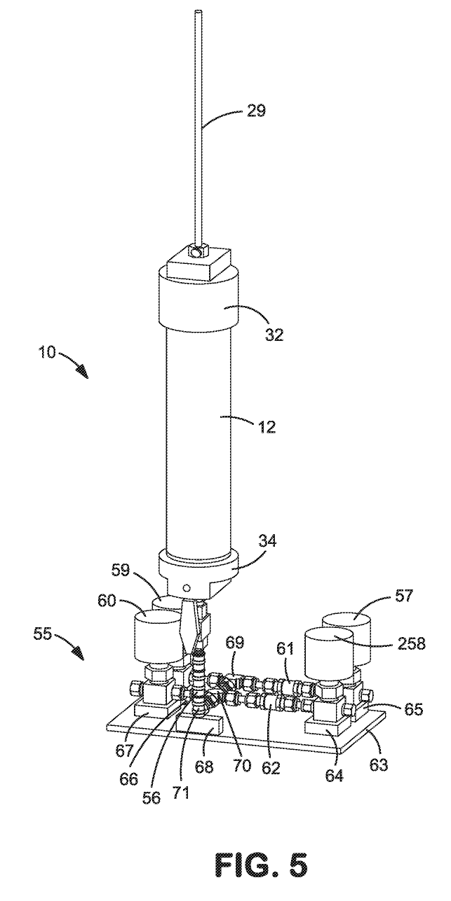

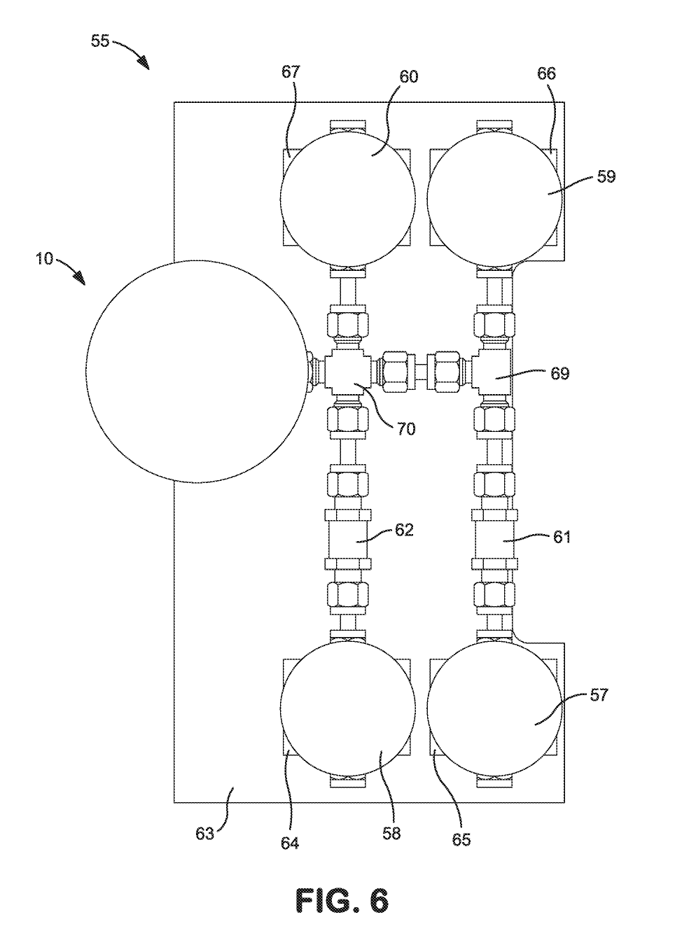

Turning now to FIGS. 5 and 6, there is shown an embodiment of a 4-valve manifold filling station 55 suitable for use in the later steps of the above-identified method (i.e. rinsing/purging and filling of cylinders or other containment vessels with a gas/liquid mixture of nitric oxide in a solvent). The cylinders or other such containment vessels 10 are preferably cleaned (e.g. rinse and purge) and then filled using the illustrated manifold filling station 55 or variation thereof. As seen in FIGS. 5 and 6, the filling and dispensing port 30 of the cylinder or vessel 10 is connected to a 316SS quick connect 56 in fluidic contact with a plurality of actuated valves 57, 58, 59, 60. In the illustrated embodiment, valve 57 is preferably connected to a source of rinsing water 157, preferably deionized and deoxygenated water. A 316SS check valve 61 prevents liquid from back-flowing into the water source 157. Valve 58 is connected to a source of nitric oxide in deionized and deoxygenated water. A check valve 62 prevents liquid from back-flowing into the nitric oxide in deionized and deoxygenated water source 158. Valve 59 is connected to a drain 159 while valve 60 is connected to a vacuum 160. The preferred 4-valve manifold filling station 55 further includes a base plate 63, valve standoffs 64, 65, 66, 67, and an elbow support 68. Fluidic communication between the valves 57, 58, 59, 60, check valves 61, 62, and the quick connect 56 is achieved using 316SS union tee 69, 316SS union cross 70, and 316SS elbow 71.

To rinse the interior chamber 14 of the cylinder or containment vessel 10, filling and dispensing port 30 and valve 57 are opened, allowing deionized and deoxygenated water to enter the first section 22 of the interior chamber 14 of the containment vessel 10, and causing the piston 25 or other moveable element to be raised in an upward direction toward the opposite end 17 of the interior chamber 14 of the containment vessel 10 until the proximal end 27 of the piston 25 touches the opposite end 17 of the interior chamber 14 of the containment vessel 10. Valve 57 is then closed and valve 59 is opened to drain the deionized and deoxygenated water from the interior chamber 14 via a purge step, described below.

To purge the cylinder or containment vessel 10, the piston 25 or other moveable element of the containment vessel 10 is lowered in a downward direction toward the dispensing end 16. Preferably, a pneumatic, hydraulic, mechanical, or electrical force is applied to the piston/rod arrangement to lower it in a downward direction toward the dispensing end 16 until the distal end 26 of the piston 25 or other moveable element is in contact with the dispensing end 16 of the interior chamber 14 of the cylinder or containment vessel 10 to purge the interior chamber of the vessel 10. Valve 60 is then opened to allow a vacuum to pull on the interior chamber of the vessel 10 and render the first section 22 of the interior chamber 14 of the containment vessel 10 generally oxygen-free.

To fill the cylinder or containment vessel with the nitric oxide solution, valve 60 is then closed and valve 58 is opened, allowing nitric oxide in deionized and deoxygenated water to enter the first section 22 of the interior chamber 14 of the containment vessel 10, and causing the piston 25 to be raised in an upward direction toward the opposite end 17 of the interior chamber 14 of the containment vessel 10.

In the preferred embodiment, a spring 26 in the second section 24 of the interior chamber 14 of the containment vessel 10 limits the movement of the piston 25 when the first section 22 of the interior chamber 14 is appropriately pressurized. Valve 58 and the filling and dispensing port 30 are then closed and the filling and dispensing port 30 of the full containment vessel 10 is removed from the quick connect 56 of the 4-valve manifold filling station 55. Because the spring 26 of the preferred embodiment will stop the movement of the piston 25 of the cylinder or containment vessel 10 when the first section 22 of the interior chamber 14 is appropriately pressurized, there is no longer a need to fill and weigh each cylinder or containment vessel 10 individually, as often required in prior art filling systems.

Although the Figs. show an embodiment with a single cylinder or containment vessel being filled, several cylinders or containment vessels 10 can be filled concurrently using an alternate embodiment or variation of the 4-valve manifold filling station 55 having multiple quick connects 56.

The initial four steps of the above-identified method, namely (1) purifying a source of water; (2) deionizing the water; (3) deoxygenating the water; (4) dissolving nitric oxide into the deionized and deoxygenated water are preferably conducted using a production system similar to that shown in FIG. 7. As seen therein, the preferred nitric oxide in deionized and deoxygenated water production system includes a water intake circuit and a holding tank constructed of PVC with stainless steel end plates configured to receive a source of water via the water intake circuit. Upstream of the holding tank in the water intake circuit there is disposed a filter and a deionizer configured to purify and deionize the water prior to delivery of the water into the tank. The end plates of the holding tank preferably include several threaded connections for the placement of inlet valves; outlet valves; vent or purge valves; and one or more sensors or analyzers.

The production system also includes one or more spargers disposed proximate the bottom of the tank and configured to sparge high purity nitrogen into the deionized water within the holding tank to deoxygenate the water within the tank. The one or more spargers are also configured to sparge high purity nitric oxide gas into the deionized and deoxygenated water within the holding tank to produce the gas/liquid mixture of nitric oxide in dissolved in deionized and deoxygenated water. An effluent of the gas/liquid mixture of nitric oxide in dissolved in deionized and deoxygenated water is then sent to a filling station where a plurality of cylinders or containment vessels are filled with the gas/liquid mixture of nitric oxide in dissolved in deionized and deoxygenated water.

The production system also includes a plurality of sensors or analyzers and a controller. The plurality of sensors or analyzers preferably includes a nitric oxide analyzer capable of measuring the concentration of nitric oxide, nitrites, and nitrates in the liquid solution as well as a dissolved oxygen sensor and pH sensor are operatively associated with the tank and couples to the controller that regulates and controls the addition of the deionized water into the tank, the flow of high purity nitrogen sparged into the tank, and the flow of high purity nitric oxide sparged into the holding tank. The controller is also configured to open and close the vent or purge valves preferably disposed at or near the top of the holding tank. Samples that are periodically withdrawn from the tank, either manually or automatically, are analyzed using the nitric oxide analyzer, with appropriate signals sent to the controller.

Specific steps must preferably be taken during use of the production system to minimize acidic byproduct formation. For example, the nitric oxide line must be purged all the way up to the tank to rid the lines of any nitrogen dioxide that may have formed, because nitrogen dioxide reacts with water to form acid byproducts. This can be achieved using a purge valve at the top of the holding tank. A flow meter placed upstream of the valve can be used for the quantification of purge amounts. If nitrogen dioxide levels in the source gas are high, the gas can be fed through a scrubber to remove the nitrogen dioxide immediately before use. Ideally, the nitric oxide line will not be purged through the tank, as any moisture on the walls of the tank may react with the nitric oxide and lead to acid formation that is difficult to fully rinse from the system. Care must be taken to release only small amounts of nitric oxide at any given time. Therefore, a good practice is to fill the lines with nitric oxide with all other valves closed, to then close the cylinder of nitric oxide, and then vent the nitric oxide lines. The nitrogen lines should also be purged to help rid the production system of any moisture that may be present. Alternatively, a vacuum system can be used to purge all lines.

Experience suggests that up to ten flushes (e.g. rinses) of the holding tank with water may be necessary, with at least the last rinse performed with deionized water. The effluent lines and outlet circuits downstream of the holding tank must also be thoroughly flushed, ideally with deionized and deoxygenated water. Stirring the water during the flushes/rinses can help to ensure adequate contact between the water and the holding tank surfaces. Stirring can also be used to encourage nitric oxide dissolution into the liquid solvent during or after the nitric oxide sparging steps.

A scrubber material can also be used to remove any nitrogen dioxide from the nitric oxide gas before it is dissolved in deionized and deoxygenated water. For example, a variety of scrubber materials can be placed downstream of the source of high purity nitric oxide gas (i.e. compressed gas cylinder of nitric oxide) for the removal of any nitrogen dioxide from the source gas, before it is dissolved in deionized and deoxygenated water within the holding tank. These scrubber materials may include, but are not limited to, calcium hydroxide (Ca(OH)2), potassium hydroxide (KOH), sodium hydroxide (NaOH), silicon dioxide (SiO2, silica), aluminum oxide (AlO3), carbon, or any mixtures thereof.

Alternatively, nitric acid formed from the presence of nitrogen dioxide in source gas may be removed from the gas/liquid mixture of nitric oxide in deionized and deoxygenated water. Removing nitric acid from the nitric oxide gas in deionized and deoxygenated water effluent can be achieved, for example using a selective membrane or by chemical reduction in the effluent circuit or outlet circuit.

Although the present inventions have been discussed with reference to one or more preferred embodiments, as would occur to those skilled in the art that numerous changes and omissions can be made without departing from the spirit and scope of the present inventions as set forth in the appended claims

What is claimed is:

1. A vessel for a gas/liquid mixture comprising nitric oxide in a liquid solvent, the vessel comprising:
a housing having a body section, a dispensing end, and an opposite end, the body section defining an interior chamber having one or more interior surfaces;
a moveable or expandable structure having a distal end, a proximal end, and one or more surfaces, the moveable or expandable structure disposed within the interior chamber to define a first section of the interior chamber configured to hold the gas/liquid mixture of nitric oxide in liquid solvent and a second section of the interior chamber configured not to hold any portion of the gas/liquid mixture of nitric oxide in liquid solvent;
wherein the dispensing end of the housing includes at least one dispensing port through which the gas/liquid mixture of nitric oxide in liquid solvent exits the first section of the interior chamber as the moveable or expandable structure moves or expands; and
wherein at least one of the one or more surfaces of the moveable or expandable structure remains in contact with a liquid surface of the gas/liquid mixture of nitric oxide in liquid solvent in the first section of the interior chamber to prevent nitric oxide from coming out of the liquid solvent.

2. The vessel of claim 1, wherein the gas/liquid mixture of nitric oxide in liquid solvent is maintained at a prescribed pressure range above ambient pressure as the volume of the gas/liquid mixture within the first section of the interior chamber changes.

3. The vessel of claim 1, wherein the gas/liquid mixture of nitric oxide in liquid solvent in the first section of the interior chamber is sealably isolated from any contaminants present in the second section of the interior chamber or from outside the housing.

4. The vessel of claim 1, wherein the moveable or expandable structure is a moveable piston or a moveable bellows having a distal end, a proximal end, and one or more side surfaces disposed within the interior chamber such that the one or more side surfaces are sealingly engaged with the one or more interior surfaces of the interior chamber to define the first section of the interior chamber between the distal end of the piston or bellows and the dispensing end of the housing and to define the second section of the interior chamber between to the distal end of the piston or bellows and the opposite end of the housing.

5. The vessel of claim 4, wherein the moveable piston or moveable bellows traverses the interior chamber toward the dispensing end of the housing in response to a mechanical force applied to or transferred to the moveable piston or the moveable bellows.

6. The vessel of claim 1, wherein the moveable or expandable structure is an expandable bellows or an expandable bladder and wherein the one or more surfaces includes an expandable surface defining an expandable interior space that defines the second section of the interior chamber.

7. The vessel of claim 2, wherein the second section of the interior chamber is configured to receive a pneumatic or hydraulic fluid to maintain the gas/liquid mixture of nitric oxide in liquid solvent in the first section of the interior chamber at the prescribed pressure range above ambient pressure.

8. The vessel of claim 2, wherein the second section of the interior chamber is configured to receive a pneumatic or hydraulic fluid to move or expand the moveable or expandable structure and dispense the gas/liquid mixture of nitric oxide in liquid solvent through the dispensing port.

9. The vessel of claim 1, wherein the housing, including components thereof and the moveable or expandable structure are constructed of materials selected from the group consisting of polyvinylchloride, aluminum, stainless steel, and fluoropolymer elastomers.

10. The vessel of claim 1, wherein one or more characteristics of the gas/liquid mixture of nitric oxide in liquid solvent contained in the first section of the interior chamber are stored in a processor operatively coupled to the vessel.

11. The vessel of claim 10, further comprising a temperature sensor or a pressure sensor and wherein the sensed pressure or sensed temperature of the gas/liquid mixture of nitric oxide in liquid solvent contained in the first section of the interior chamber are displayed on a gauge or a display attached to an exterior surface of the vessel.

12. The vessel of claim 10, wherein the volume of the gas/liquid mixture of nitric oxide in liquid solvent contained in the first section of the interior chamber are displayed on a gauge or a display attached to an exterior surface of the vessel.

13. The vessel of claim 12, further comprising an alarm when the pressure of the gas/liquid mixture of nitric oxide in liquid solvent contained in the first section of the interior chamber is outside a predetermined pressure range or below a pressure threshold.

14. The vessel of claim 12, further comprising an alarm when the temperature of the gas/liquid mixture of nitric oxide in liquid solvent contained in the first section of the interior chamber is outside a predetermined temperature range or temperature threshold.

15. The vessel of claim 12, further comprising an alarm when the volume of the gas/liquid mixture of nitric oxide in liquid solvent contained in the first section of the interior chamber is below a predetermined volume threshold.

16. The vessel of claim 10, wherein the processor is configured to store data about the gas/liquid mixture of nitric oxide in liquid solvent contained in the first section of the interior chamber selected from the group consisting of characteristics of the gas/liquid mixture of nitric oxide in liquid solvent in the first section of the interior chamber, temperature of the gas/liquid mixture of nitric oxide in liquid solvent in the first section of the interior chamber, pressure of the gas/liquid mixture of nitric oxide in liquid solvent in the first section of the interior chamber, volume of the gas/liquid mixture of nitric oxide in liquid solvent in the first section of the interior chamber, any alarm condition, or combinations thereof.

17. The vessel of claim 16, wherein the processor is further configured to transmit some or all of the data stored in the processor to another device via a wireless or wired connection.

18. The vessel of claim 1, further comprising a filling port disposed on the housing and in fluidic communication with the first section of the interior chamber and configured to permit selective filling of the gas/liquid mixture of nitric oxide in liquid solvent into the first section of the interior chamber.

19. The vessel of claim 1, further comprising a port disposed on the housing and in fluidic communication with the second section of the interior chamber and configured to permit selective filling of a pneumatic or hydraulic medium into the second section of the interior chamber and venting or release of the pneumatic or hydraulic medium from the second section of the interior chamber.

20. A method for dispensing a gas/liquid mixture comprising nitric oxide in liquid solvent from a vessel comprising the steps of:
(a) purifying a source of water;
(b) deionizing the water;
(c) deoxygenating the water;
(d) dissolving nitric oxide into the deionized and deoxygenated water;
(e) rinsing/purging a plurality of cylinders, cartridges or other containment vessels; and
(f) filling a plurality of cylinders, cartridges or other containment vessels with the gas/liquid mixture of nitric oxide in deionized and deoxygenated water.

21. A dispensing system for a gas/liquid mixture comprising:
a container comprising a body section defining an interior chamber having one or more interior surfaces; a dispensing end; and an opposite end;
a moveable or expandable structure disposed within the interior chamber of the container, the moveable or expandable structure having a distal end, a proximal end, and one or more side surfaces, the moveable or expandable structure configured such that the one or more side surfaces are sealingly engaged with the one or more interior surfaces of the interior chamber of the container to define a first section of the interior chamber between to the distal end of the moveable or expandable structure and dispensing end of the container and a second section of the interior chamber between to the proximal end of the moveable or expandable structure and the opposite end of the container;
the gas/liquid mixture comprising nitric oxide gas in liquid solvent disposed in the first section of the interior chamber of the container; and
a motive source in operative association with the container and the moveable or expandable structure and configured to cause the moveable or expandable structure to traverse the interior chamber of the container;
wherein the dispensing end of the container includes at least one port through which the gas/liquid mixture of nitric oxide in liquid solvent is dispensed as the moveable or expandable structure traverses or expands within the interior chamber of the container; and
wherein the distal end of the moveable or expandable structure remains in contact with a liquid surface of the gas/liquid mixture of nitric oxide in liquid solvent in the first section of the interior chamber so as to prevent nitric oxide from coming out of the liquid solvent.

22. The dispensing system of claim 21 wherein the gas/liquid mixture of nitric oxide in liquid solvent is maintained at a prescribed pressure range above ambient pressure as the volume of the gas/liquid mixture within the first section of the interior chamber changes.

23. The dispensing system of claim 21 wherein the gas/liquid mixture of nitric oxide in liquid solvent in the first section of the interior chamber is sealably isolated from any contaminants present in the second section of the interior chamber or from outside the container.
